(19) United States
(12) Patent Application Publication
(10) Pub. No.: US 2026/0345016 A1
(43) Pub. Date: Nov. 5, 2026

(54) A PHOTOPLETHYSMOGRAPHY SYSTEM AND METHOD

(71) Applicant: IMPERIAL COLLEGE INNOVATIONS LIMITED, London (GB)

(72) Inventors: Stefan Karolcik, London (GB); Pantelis Georgiou, London (GB)

(21) Appl. No.: 19/136,466

(22) PCT Filed: Dec. 5, 2023
(86) PCT No.: PCT/EP2023/084228

§ 371 Date: Jun. 6, 2025

(30) Foreign Application Priority Data
Dec. 7, 2022 (GB) 2218359.4

(51) Int. Cl. A61B 5/021; A61B 5/00; A61B 5/0205; A61B 5/024; A61B 5/11; A61B 5/145; A61B 5/1455

(52) U.S. Cl. CPC A61B 5/02125; A61B 5/02055; A61B 5/02416; A61B 5/02438; A61B 5/7278; A61B 5/7282; A61B 5/746; A61B 5/1126; A61B 5/14535; A61B 5/14551; A61B 2560/0223; A61B 2562/0219

(57) ABSTRACT

Disclosed herein is a photoplethysmography (PPG) system for health monitoring. The system comprises a first sensor array configured to collect first PPG data and configured for attachment to a patient at a first sensing site; a second senor array configured to collect second PPG data and configured for attachment to the patient at a second sensing site; and one or more processors configured to receive the first and second PPG data and determine, based on the first and second PPG data and a known distance between the first and second sensing site, a blood pressure value for the patient.

This disclosure relates to a photoplethysmography system, and in particular to a photoplethysmography system for health monitoring.

### BACKGROUND

Photoplethysmography (abbreviated as PPG) has been applied in clinical and fitness health monitoring. Established commercial applications of photoplethysmography are primarily limited to fitness heart-rate monitoring using smartwatches and smartphones, and fingertip pulse oximetry in clinical settings.

Separately, there is a need for mass-deployable and non-invasive systems for health monitoring, for example in regions suffering from seasonal epidemics. Due to nature of these diseases, there is a significant strain on the healthcare sector caused by short-term rapid infection spread. For example, dengue infects over 100 million people annually in tropical regions. Even with only 1-5% of the cases developing into severe illness, it creates a large cohort of patients requiring urgent medical care. The current methods of patient triage are limited, for example by staff capacity, which can lead to missed severe cases during the peak infection season. Existing PPG systems are not suitable to meet this need, in part because measuring a patient's heart rate and/or oxygen level in the blood only provides limited information about the patient's health. In summary, existing PPG systems cannot meet this need because they suffer from one or more of the following disadvantages: they are expensive, cannot be mass-produced, require a clinical setting, are uncomfortable to wear for longer periods of time or are otherwise not suitable for continuous monitoring, and/or are limited in their functionality.

The present invention seeks to address these and other disadvantages encountered in the prior art by providing an improved PPG system for health monitoring.

### SUMMARY

According to an aspect, there is provided a photoplethysmography (PPG) system for health monitoring. The system comprises a first sensor array configured to collect first PPG data and configured for attachment to a patient at a first sensing site, and a second senor array configured to collect second PPG data and configured for attachment to the patient at a second sensing site. The system also comprises one or more processors configured to receive the first and second PPG data and determine, based on the first and second PPG data and a known distance between the first and second sensing site, a blood pressure value for the patient.

Determining the blood pressure value for the patient may be further based on a time lag between first waveforms in the first PPG data and second waveforms in the second PPG data.

Determining the blood pressure value for the patient may comprise determining the time lag, determining a pulse wave velocity value for the patient based on the determined time lag and the known distance between the first and second sensing site, and determining the blood pressure value using the determined pulse wave velocity and one or more calibration coefficients.

The first sensor array may be configured to collect the first PPG data in a first plurality of wavelength channels. The second sensor array may be configured to collect the second PPG data in a second plurality of wavelength channels. The first and second plurality of wavelength channels may each comprise at least three wavelength channels. The second plurality of wavelength channels may comprise at least two wavelengths which define an isosbestic point for Haemoglobin (HB) and Oxy-haemoglobin (HbO2). The at least two wavelengths may be substantially, or exactly, 800 nm and 1300 nm.

The one or more processors may be further configured to determine, based on the first and second PPG data, a haematocrit value for the patient.

The one or more processors may be further configured to monitor one or more health parameters comprising blood pressure, haematocrit value, heart rate, and oxygen saturation in the blood. The one or more processors may be further configured to compare the one or more health parameters to a baseline value for the patient and, if the one or more health parameters differs from the baseline by more than a threshold value, triggering an alert.

The system may comprise a housing for the first sensor array, the housing coupled to a wrist strap for positioning the first sensor array against a wrist of the patient.

The system may comprise a ring or finger clip which comprises the second sensor array, for positioning the second sensor array against a finger of the patient.

The one or more processors may be coupled to each of the first and second sensor arrays.

The system may be wearable.

The one or more processor may comprise a first processor and a server, for example. The system may then comprise a wearable device which in turn comprises the first and second sensor array and the first processor. The first processor may be configured to receive the first and second PPG data and communicate it, e.g. transmit it via a wired or wireless connection, to the server. The server may be configured to determine the blood pressure value for the patient.

The system may comprise a temperature sensor configured to monitor a temperature of the patient; and the processor may be further configured to determine if the patient's temperature changes from a baseline temperature for the patient by a threshold amount.

The system may comprise an accelerometer configured to monitor a motion of the patient, and the processor may be further configured to determine if the patient's motion changes from a baseline motion for the patient by a threshold amount.

According to another aspect there is provided a computer-implemented method for use with the system of any preceding claim. The method comprises receiving first PPG data obtained from a first sensor array attached to a first sensing site of a patient, and receiving second PPG data obtained from a second sensor array attached to a second sensing site of the patient. The method further comprises determining, based on the first and second PPG data and a known distance between the first and second sensing site, a blood pressure value for the patient.

Determining the blood pressure value for the patient may be further based on a time lag between first waveforms in the first PPG data and second waveforms in the second PPG data. Determining the blood pressure value for the patient may comprise determining the time lag; determining a pulse wave velocity value for the patient based on the determined time lag and the known distance between the first and second sensing site; and determining the blood pressure value using the determined pulse wave velocity and one or more calibration coefficients.

The method may comprise determining, based on the first and second PPG data, a haematocrit value for the patient.

The method may comprise monitoring one or more health parameters comprising blood pressure, haematocrit value, heart rate, and oxygen saturation in the blood.

The method may comprise comparing the one or more health parameters to a baseline value for the patient and, if the one or more health parameters differs from the baseline by more than a threshold value, triggering an alert.

According to another aspect there is provided a computer readable medium comprising instructions which, when implemented by a processor, cause the processor to perform one or more of the methods disclosed herein.

### FIGURES

Specific implementations are now described, by way of example only, with reference to the drawings, in which:

FIG. 1 depicts a PPG waveform shape and commonly extracted features.

FIG. 2 depicts light absorption properties for various blood components and water across the visible and near-IR light spectrum.

FIG. 3 depicts a system according to the present disclosure.

FIGS. 4a and 4b depict components of a wrist sensor according to a specific implementation of a first sensor array, according to the present disclosure.

FIGS. 5a and 5b depict components of a finger sensor according to a specific implementation of a second sensor array, according to the present disclosure.

FIG. 6 depicts a block diagram of the system of the present disclosure.

FIG. 7 depicts a digital signal processing pipeline which may be used on the raw data obtained from the system of the present disclosure.

FIG. 8 depicts a method according to the present disclosure.

FIGS. 9a-e depict waveforms from an experiment(s) using the system of the present disclosure.

FIGS. 10a-d depict results from an experiment(s) using the system of the present disclosure.

FIG. 11 depicts a computer system which may be used to implement one or more methods of the present disclosure.

### DETAILED DESCRIPTION

In overview, and without limitation, the application discloses a photoplethysmography (PPG) system for health monitoring. The system comprises two different sensor arrays, both configured to collect PPG data, and configured for positioning at two different sensor sites on a patient's body. As an example, the sensor sites may be the patient's finger, and the patient's wrist. By making use of two different sensor sites in this way, a processor coupled to the sensor arrays is able to determine, based on the PPG data received from the sensor arrays and a known distance between the first and second sensing site, a blood pressure value for the patient.

In an example implementation, the system is able to continuously monitor the patient's blood pressure, in addition to other health parameters such as haematocrit value, heart rate, and oxygen saturation in the blood. If one or more of these parameters deviates from a baseline value for the patient, for example by more than a threshold amount, then the system may alert the patient or healthcare providers in an appropriate manner.

The presently disclosed system is therefore able to alleviate the pressure on the healthcare sector by providing a low-cost, continuous monitoring alternative to early symptoms hospitalisation. This way, only the truly severe cases, as indicated by a significant change in sensing parameters, will require medical intervention. The system may be a modular PPG acquisition platform that combines an array of wavelengths and two sensing sites to obtain non-invasive estimates of multiple biological parameters. Due to its modular design, the sensory probes can be changed according to the requirements of the patient. The platform has been designed to allow synchronised acquisition from multiple body locations in parallel.

A specific use case for the system is monitoring of health parameters relevant to Dengue. Highly infectious disease like dengue can be tackled more effectively by having a low-cost, non-invasive, continuously monitoring alternative for patient deterioration detection. However the present system and associated methods are by no means limited to this use case. On the contrary, the present system provides a base for complex PPG model development and validation, beyond the usual pulse oximeter or heart-rate monitoring applications.

PPG Sensing and Vital Sign Estimation from PPG Data

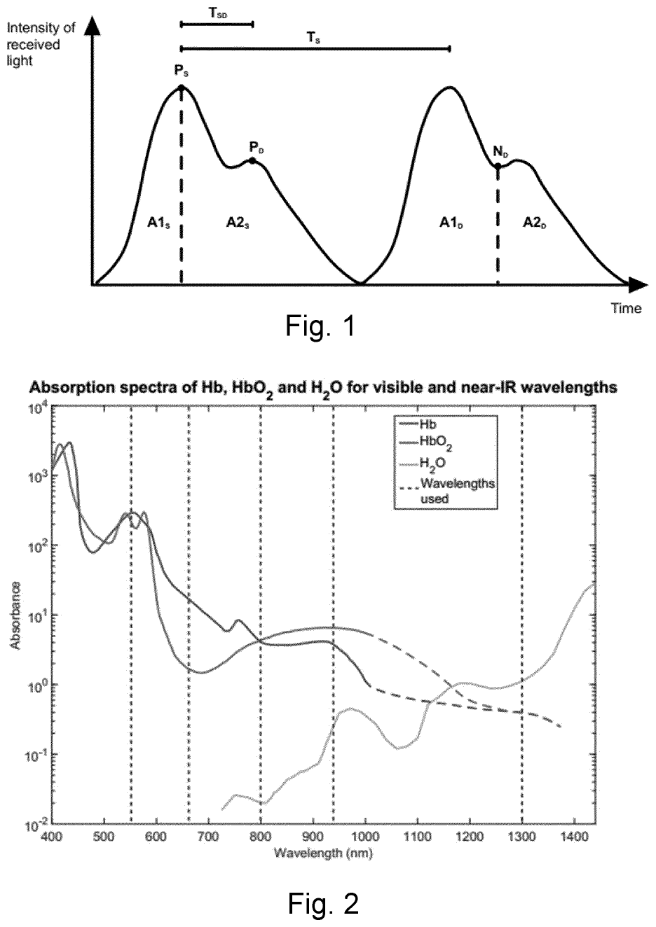

FIG. 1 depicts a PPG waveform shape and some commonly extracted features. The graph shows “intensity of received light” on the y axis, and time on the x axis. The graph shows two periods of the waveform with clearly defined dicrotic notch that is acquired from a fingertip sensor. The common features include: TSD: Time period between systolic and diastolic peak during a single beat, also related to large artery stiffness index; TS: Time period between two consecutive systolic peaks, also corresponding to the heart rate and heart rate variability when measured over longer periods; PS: Systolic peak; PD: Diastolic peak; ND: Dicrotic notch, the slight increase in the pressure in the beginning of diastole caused by the closure of the aortic valve; A1S-A2S: Area under the curve split by the systolic peak; A1D-A2D: Area under the curve split by the dicrotic notch.

FIG. 2 shows light absorption properties for various blood components and water across the visible and near-IR light spectrum. In particular, the figure shows absorption spectra for Haemoglobin (Hb), Oxy-Haemoglobin (HbO₂), and water (H₂O) for visible and near-IR wavelengths. The graph shows absorbance on the y axis measured in cm⁻¹, and wavelength on the x axis measured in nm. The solid lines depict absorbance as a function of wavelength, and dashed continuation of lines correspond to the approximate absorbances published in literature. The vertical horizontal lines correspond to particularly beneficial wavelengths which may be used by the present system (described in greater detail below).

### PPG

A photoplethysmogram is obtained by illuminating skin using a light source at visible or near-IR wavelength and capturing the transmitted light with a photodiode. As light travels through the tissue, multiple absorption and scattering events occur, allowing the extraction of vital biological information on the receiver side. When sensing PPG, for example on a fingertip or a wrist, most parts of the tissue are static and therefore their absorption can be approximated as constant provided the subject/patient is at rest. The constant absorption then manifests as a DC offset in the resultant PPG waveform. The main contributor to the alternating part of the PPG waveform, pictured in FIG. 1, is the periodic blood flow caused by heart activity. Every time the heart pushes blood through the body arteries, the volume of blood at sensed site momentarily changes, creating peaks and troughs in the waveform. This allows direct extraction of heart-beat provided the peaks are clearly distinguishable. It is important to note that PPG systems are notably susceptible to motion artefacts as the initial approximation of constant absorption of surrounding tissue is no longer valid when subject is moving.

Conventional PPG systems are typically built with a particular application in mind, with optimised acquisition circuitry for either heart-rate or oxygen saturation measurement. The receiver pipeline may consist of a photodiode connected to a trans-impedance amplifier, followed by a gain stage and an analogue-to-digital converter with optional analogue filtering on the way.

### Light Absorption

PPG analysis of more than the heart-beat is possible thanks to the varying absorptivity of light wavelengths across the visible and IR spectrum with respect to blood constituents. This approach can be explained by the Beer-Lambert Law of Absorbance, applied to the blood vessels with the following equation:

A=ε(λ)lc (1)

where A is the absorbance, ε(λ) is the absorption coefficient of the component in the measure sample at specific wavelength (λ), l is the optical path length through the sample and c is the concentration of the component in the measured sample.

FIG. 2 shows the varying absorbance of blood constituents at different wavelengths. Combining specific wavelengths in a single sensor probe at similar or identical optical path lengths, allows the cancellation of parts of the Beer-Lambert equation to obtain the concentration estimate for a given constituent.

Estimation of Biological Parameters from PPG

Established commercial applications of photoplethysmography are limited to fitness heart-rate monitoring using smartwatches and smartphones and fingertip pulse oximetry in clinical settings.

In contrast with these known systems, the functionality of the present system is significantly extended. The present disclosure relates to a system capable of determining (or at least estimating) and monitoring health parameters, including a patient's blood pressure and/or haematocrit. The present inventors expect that providing a blood pressure estimation, without a pressure cuff, using an inexpensive and non-invasive sensor based on PPG in the present manner will have a major impact on tackling one of the most widespread illnesses in the world: hypertension (high blood pressure).

Reference is made herein to “blood pressure”. Clinicians, especially within the context of dengue-related shock, often refer to “Pulse Pressure”. This is directly extracted from blood pressure, and Pulse Pressure=systolic BP-diastolic BP. The skilled person will appreciate that, while reference is primarily made to blood pressure herein, the present system and method may equally determine/estimate pulse pressure according to the use case to which the wearable system is being applied.
1) Heart rate (HR): By inspection of FIG. 1, it can be appreciated that the heart-rate peaks (Pₛ, P_{D}) are the most prominent features of the PPG waveform. Therefore, the only parameter needed to calculate HR is sampling frequency. By not requiring to capture any more features except the peaks, a green wavelength may be chosen as it does not penetrate the tissue as deep as longer wavelengths, and therefore is more resistant to motion artefacts at the cost of being less feature rich.
2) Pulse oxymetry (SpO2): In clinical settings, PPG is used to measure oxygen saturation in blood also known as pulse oximetry or SpO2 measurement. Oxygen saturation is calculated directly from ratios between PPG signal waveforms at two distinct wavelengths. The two values are selected to maximize the difference between absorption in oxygenated hemoglobin (red blood cells that carry oxygen) and deoxygenated hemoglobin (red blood cells not carrying oxygen). Suitable wavelengths include 660 nm (red) and 940 nm (infrared). Looking at FIG. 2, a clear difference in absorption can be observed, with the red wavelength being dominated by Hb and the infrared being dominated by Hbo2. A ratio R is calculated using the formula below:

$$R \approx \frac{AC ( {λ}_{1} ) / DC ( {λ}_{1} )}{AC ( {λ}_{2} ) / DC ( {λ}_{2} )} \qquad ( 2 )$$

where AC(λ) is the pulsatile component of the PPG waveform at given wavelength λ and DC(λ) is the DC offset of the PPG waveform. This ratio R can be directly used to obtain an SpO2 value using a calibration curve.
3) Haematocrit (Hct): The theory behind Hct sensing is based on a similar principle to that of pulse oximetry. Haematocrit may be defined as the proportion of red blood cells in whole blood. The process can be simplified using an approximation. By approximating the rest of the blood's constituents (plasma) to water in terms of light absorption, a Hct value can be determined from the ratio between red blood cells and water. It has been determined that optimal wavelengths selected to carry out monitoring of a patient's Hct are 800 nm and 1300 nm (as shown in FIG. 2). These values are chosen to be at the isosbestic point, i.e. the point at which light absorption is the same for Hb and HbO2. This means that varying values of SpO2 will not interfere with the measurement. The main downfall of using this prior method is that at 1300 nm, the absorption of water is the dominant factor and water is present across the human body, not only in blood which highly attenuates the transmitted light intensity. The present method addresses this issue by providing multiple wavelength channels, including the isosbestic wavelengths (800 and 1300) and two additional wavelengths to increase the performance of the device and provide a more accurate determination of Haematocrit.
4) Blood pressure (BP): Estimating blood pressure from PPG has been attempted by a number of techniques with varying accuracy and results. To date, it has been thought that to obtain an accurate determination, it is necessary to not use the PPG signal alone, but to additionally employ a wider variety of bio-signals, mainly ECG. To estimate the blood pressure from combination of PPG and ECG, a surrogate called Pulse transmit time, or PTT, is extracted from the data. PTT is defined as the time difference between the peaks of PPG and ECG waveforms and it has been shown to correlate well with blood pressure despite the requirements on frequent re-calibration. However, building a system around PTT brings additional design challenges making the system more complicated. Requiring electrical coupling to the patient for ECG electrodes and multiple contact points on the body makes the system uncomfortable to wear for non-critical patients.

The present method avoids these design issues. As will be described in greater detail below, a first and a second sensor array may be positioned a known (e.g. a measured) distance apart on the patient, and a processor can then determine, based on the PPG data produced by the sensor arrays and the known distance between the first and second sensing site, a blood pressure value for the patient. Determining the blood pressure value for the patient is further based on a time lag between first waveforms in the first PPG data and second waveforms in the second PPG data.

In more detail, the two PPG sensors spaced a known distance apart can be used to calculate pulse wave velocity (PWV), and in turn convert to blood pressure using the equation:

P=k₁ ln(c²)+k₂ (3)

where P is the blood pressure, c is the pulse wave velocity and k1,2 are calibration constants that may be obtained during calibration. These calibration constants depend on many factors; a simple way to obtain them is to take a couple of blood pressure readings from another means, e.g. using a cuff-based device, when a system and/or device according to the present disclosure is first given to the patient and calculate the constants from those measurements.

Using a machine learning method described below, blood pressure may be estimated from PPG waveform data alone, using its rich, previously underutilised feature set. Looking back at the FIG. 2, the combination of some of the illustrated features may be successfully used to train neural networks for BP estimation from PPG data.

The present disclosure will start by explaining the extraction of the PWV surrogate first, before describing a machine learning approach below.

### The System

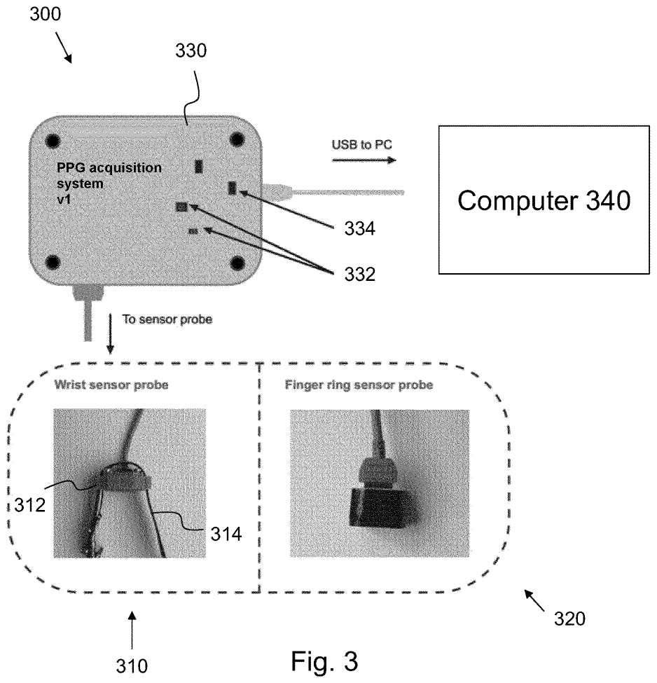

FIG. 3 depicts an illustration of a system 300 according to the present disclosure. The system 300 comprises a first sensor array 310, which may be described as a first probe. The system 300 further comprises a second sensor array 320, which may be described as a first probe. The system 300 also comprises a data acquisition unit 320, and a computer 340 or other suitable data processing unit.

The first sensor array 310 is configured to collect first PPG data. The first sensor array 310 is configured for attachment to a patient at a first sensing site. In the implementation shown, the first sensor array 310 is designed and configured for attachment to a patient's wrist. The system 300 comprises a housing 312 for the first sensor array 310, the housing 312 coupled to a wrist strap 314 for positioning the first sensor array against a wrist of the patient. The sensors of the first sensor array 310 are mounted to the housing 312.

The second sensor array 320 is configured to collect second PPG data. The second sensor array 320 is configured for attachment to a patient at a second sensing site. The system 300 comprises finger attachment means, in particular a ring (or in other, non-depicted implementations, a finger clip) which comprises the second sensor array 320. The sensors of the second sensor array 320 are mounted to the finger attachment means such that the sensor array 320 may be positioned against a finger of the patient.

The system 300 comprises a data acquisition unit 330 and a computer 340. The data acquisition unit 330 is coupled to both the first and second sensor arrays 310, 320, and is configured to receive the PPG data. This coupling may be achieved by a wired coupling, as shown, or a suitable wireless coupling that allows the transfer of data. In an example implementation, the data acquisition unit 330 contains one or more AFE4900EVM boards. These boards are produced by Texas Instruments™ abbreviated as TI, and provide an analog front end (AFE) for PPG acquisition and analogue-to-digital conversion of the acquired data. Custom-designed probes with standardized 10-pin connector are connected on one side, while a microUSB cable is connected to the computer 340 from the other side. Two USB cables and two analogue cables are used to operate the two sensor probes 310, 320 in parallel. The data acquisition unit 330 comprises two status LEDs 332 and a reset switch 334. The computer 340 comprises an application GUI capable of showing a real-time data stream coming from the sensor arrays 310, 320, from all 8 wavelength channels (as will be defined later).

In a specific implementation, the data acquisition unit 330 comprises two AFE4900 boards, each equipped with 4 independent LED channels and up to 3 independent photodiode inputs. A range of configuration options allows each of the LED channels to be separately configured for variable gain, DC offset current and LED forward current. The platform can therefore support a large selection of off-the-shelf LED's and photodiode pairs at varying wavelengths. All settings are stored in internal registers of the chip, configured via SPI. For the first iteration of the platform, a modified version of the development board provided by TI was used. Miniaturisation is another avenue provided by this particular chip range selection. The whole system 300 has been carefully designed to not require any physically large components to enable a wearable implementation.

To allow data acquisition from multiple body sites, the system 300 contains two of these development boards each connected to a custom made sensor probe 320. In total, 5 distinct wavelengths and 8 distinct PPG channels are acquired in parallel. The pulse oximetry wavelengths (660 nm and 940 nm) are present on both probes but due to high noise impact on the reflectance wrist probe, only the transmissive fingertip signal is used in experiments. In total, the second sensor array 320 in the form of a transmissive finger probe, contains 4 wavelengths: 660 nm, 800 nm, 940 nm and 1300 nm—corresponding to the wavelength for pulse oximetry and haematocrit sensing combined. The first sensor array 310, in the form of a reflective wristwatch style probe, contains 3 wavelengths and an ambient channel: 525 nm, 660 nm and 940 nm. Here, only the green channel PPG waveform is used to calculate pulse velocity across the hand and confirm heart-rate readings.

It will be appreciated by the skilled person that while figures depict a proto-type system which comprises both a data acquisition unit 330 and a computer 340, the system 300 may instead comprise a single processing unit which combines the functionalities demonstrated by these separate units. In particular, methods of the present disclosure can be performed directly on a suitably configured data acquisition unit 330 without the need for a separate data processing unit. This data acquisition unit 330 may be directly coupled to the housing 312, and thus the entire system 300 may be completely and entirely wearable by a patient/user. In an implementation, the wearable system comprises a first and second sensor array complete with suitable housings and components to allow attachment to the patient, a processor, and a battery unit configured to provide power to the other components of the system.

### Probe/Sensor Array Design and Location

The design requirements for probes configured for different sensing sites include the shape and feature set of the PPG waveform, and therefore care needs to be taken when selecting the correct sensing sites for the given application. For the dengue use case, requirements for the locations were as follows: comfortable to wear for long periods of time, acceptable quality PPG signal available and close proximity of the two locations. Fingertip and wrist were picked as optimal spots for measurements for this use case, however the skilled person will appreciate that the present system and method(s) are generally applicable to different sensing sites. For example, depending on the use case, it is possible to design a system configured to collect data from a patient's earlobe, forehead, neck (main artery), wrist, fingers and/or toes.

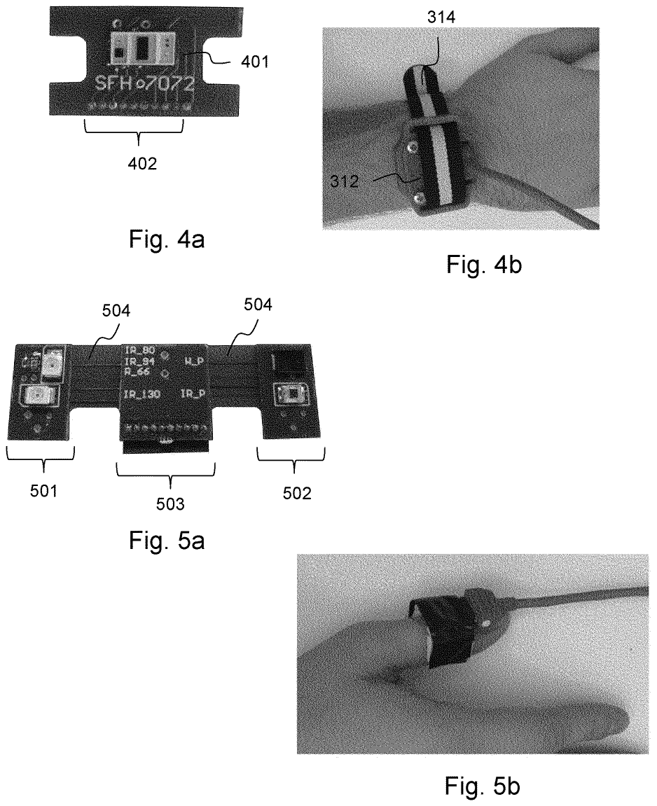

FIGS. 4a and 4b depict a specific implementation of the first sensor array. In this implementation, it takes the form of a wrist probe design inspired by a wristwatch. The wristwatch design can be appreciated by inspection of FIG. 4b. The FIG. 4b shows the housing 312 and strap 314. The housing 312 houses the PCB which can be seen in FIG. 4a. The wristwatch housing 312 houses the PCB inside a 3D printed structure secured on hand by using the flexible strap 312. The 10-lead medical cable is directly connected to the probe. The PCB pictured in 4a contains the PPG array sensor SFH7072 from OSRAM 401 and a 10-lead connector 402.

A watch like design allows comfortable wear while providing good adhesion to the skin. The probe casing is custom 3D printed. The PCB inside contains an off-the-shelf reflectance PPG LED array SFH7072 (OSRAM) containing the 3 wavelengths: 525 nm, 660 nm and 940 nm with one broadband photodiode and one IR-cut photodiode with improved sensitivity to visible wavelengths. Standard wrist-watch style band allows the accommodation of various wrist sizes.

FIGS. 5a and 5b depict a specific implementation of the second sensor array. In this implementation, it takes the form of a ring probe suitable for placement over a patient's finger. Fingertip sensors for clinical use are less suitable for constant monitoring as they completely obscure the end of the finger and make the hand hard to use. Therefore, in some implementations, an alternative, ring-like design is utilised.

The semi-flex PCB design of the ring probe provides tight fit on variety of finger shapes and thicknesses. The sensor array should be placed on the top part of the finger or directly on the fingertip. The pictured PCB design in 5a contains an emitter region 501, detector region 502, 10-lead connector 503 and flexible regions 504 allowing 90 degree bends.

The finger probe design utilizes the semi-flex PCB technology to accommodate various finger sizes. As pictured in FIG. 5a the board consists of 3 rigid parts and 2 flexible interconnects that allow the PCB to loop around the finger. A total of 4 medical grade SMT package LEDs are used on one side to provide the emitting part of the PPG sensor. There are two photo-diodes on the receiver side, the first one being a standard broadband photodiode sensitive to light wavelengths from 500 nm to 1000 nm. The second is an InGaAs photodiode sensitive in the 1300 nm wavelength region paired with the deep IR LED. The PCB is encompassed in a flexible, rubber-like 3D printed material to allow tight fit on the finger. The electronically sensitive parts of the PCB are sealed in non-conducting epoxy and covered by a translucent plastic film to prevent shorts caused by sweat or accidental liquid spillage.

It should be understood that the device described in the above paragraph, and in relation to FIGS. 4a,b and 5a,b, is not the only way to implement the functionality described in the present application, but is instead simply one way of implementing the methodologies disclosed herein.

### The Acquisition Platform

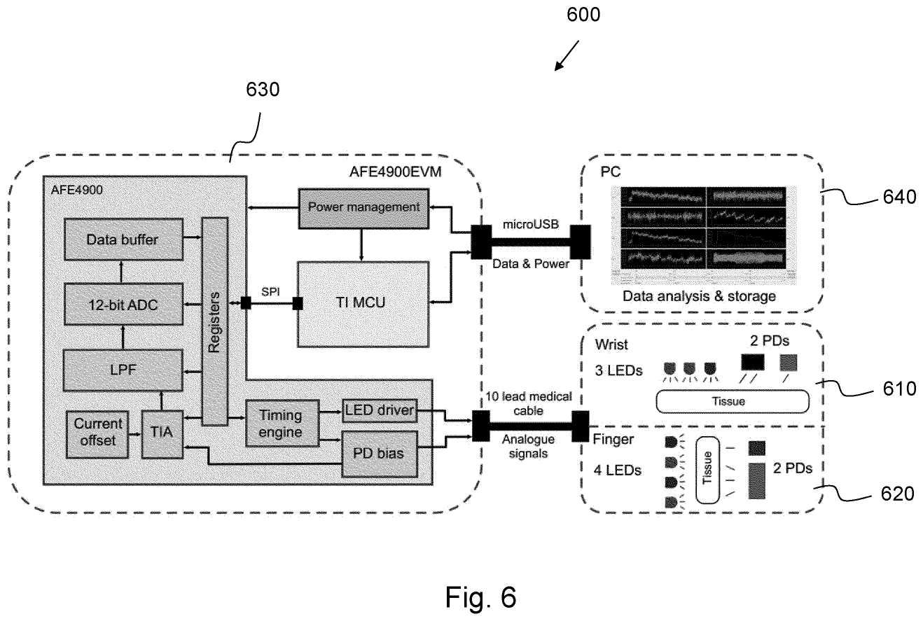

FIG. 6 depicts a block diagram of a PPG acquisition system 600 according to the present disclosure. The system 600 may be referred to as a ‘platform’ elsewhere herein. As described above in relation to FIG. 3, the system comprises a first and a second sensor array/probe 610, 620; a data acquisition unit 630, and a computer 640 (labelled here as a PC). The data acquisition unit 630 comprises two AFE4900EVM development boards from Texas Instruments™, which are each built around the AFE4900 analogue front-end chip for PPG (only a single development board is depicted in FIG. 6 for clarity of illustration). A first of these chips is associated with the first sensor probe 610, and a second of these chips is associated with a second sensor probe 620. Each chip comprises a PD bias unit, an LED driver, a timing engine, a transimpedance amplifier (TIA), a current offset unit, a low pass filter (LPF), a 12-bit ADC, a data buffer unit, and registers. Each chip contains the whole pipeline for the PPG signal with most of the block being configurable by internal registers. To configure this chip and retrieve data, an intermediary microcontroller (labelled TI MCU in FIG. 6) translates the standard serial commands from the PC 640. The probes 610, 620 utilise a 10-lead analogue medical cable with adequate shielding to prevent cross talk. The wrist probe 610 contains 3 LEDs and 2 photodiodes (PDs) in reflectance configuration, while the finger probe 620 contains 4 LEDs and 2 PDs in transmissive configuration.

The block diagram in FIG. 6 illustrates the 3 main parts of the system 600. The analogue front-end chip of the data acquisition unit 630 contains all necessary circuitry to acquire a PPG waveform. On the emitter side, the configurable LED driver is used to drive up to 4 separate LEDs in parallel. The current going through each LED can be adjusted to values in the range of 0 to 200 mA. On the receiver side, the photodiode is fed directly into a trans-impedance amplifier stage with an optional DC current offset which is then low-pass filtered and digitised. The chip is capable of having 3 different photodiodes connected at the same time and multiplexes between them based on the timing controls. The timing registers inside of the AFE4900 guide the frequency of acquisition and define which LED should be paired with which photodiode. Each LED is only turned on for a fraction of the PPG acquisition period to preserve power and allow all 4 channels in a single cycle. The LED “ON” time can be configured and it is determined based on the selected photodiode settling time.

The acquired raw data for all 4 channels is digitised using a 12-bit ADC and stored in internal registers. The 12-bit values are extracted from the board via an embedded microcontroller implementing an USB serial port communication to PC. At the other end, the 10-lead medical cable connects to the sensor probe where the pins correspond to either an LED driver, photodiode biasing receiver or ground connection. The modularity of the design allows creation of new probes as long as the pin order of the connector is maintained and the sensing components are rated below the maximum ratings of the analogue chip. This way, any new probe design only requires a software configuration before it can be used for raw PPG acquisition.

The finished device is encased in a custom case with 2 AFE4900EVM boards stacked on top of each other, with the case as depicted in FIG. 3. Each sensor probe may be operated independently, connected to a separate TI board. The data is then synchronized in the accompanying software application after being received by the PC from two USB cables.

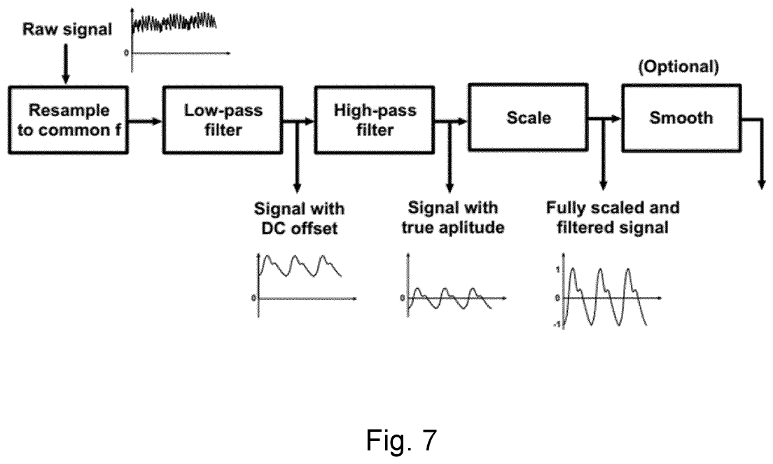

FIG. 7 depicts a digital signal processing pipeline used on the raw data obtained from the system 600. In the first stage, labelled “resample to common f”, signals from the wrist probe are re-sampled to match the finger probe frequency and synchronised. The second stage, labelled “low-pass filter”, employs a low pass filter with a cutoff frequency of 20 Hz that removes the high frequency noise including the 50 Hz mains interference. In the next stage, labelled “high-pass filter”, a high-pass filter with a cutoff frequency of 0.5 Hz removes the DC offset. Lastly, the scaling step, labelled “scale”, allows precise morphology-based feature extraction. This step is only applicable for features that do not depend on true amplitude. An optional final step, labelled “smooth”, of smoothing is sometimes performed for low amplitude signals.

One of the key elements of the presently disclosed system is synchronized data acquisition with up to 8 separate PPG waveforms and raw data export for further analysis. To achieve this, a custom multithreaded Windows™ application can be developed. A graphical user interface (GUI) may be written in Python to visualize all 8 possible LED channels with waveform data in real-time. In the backend, the system may keep reading both serial ports in parallel using separate software threads and assigns timestamps to datapoints as they come. Timestamps are saved together with the data to allow synchronisation between the two boards even if their internal clocks are not matched. Saving to file is incremental, protecting the system from complete data loss in case the system would stop responding for any reason. Communication between the board and PC is implemented via a TI serial protocol customized for parallel multi-board acquisition.

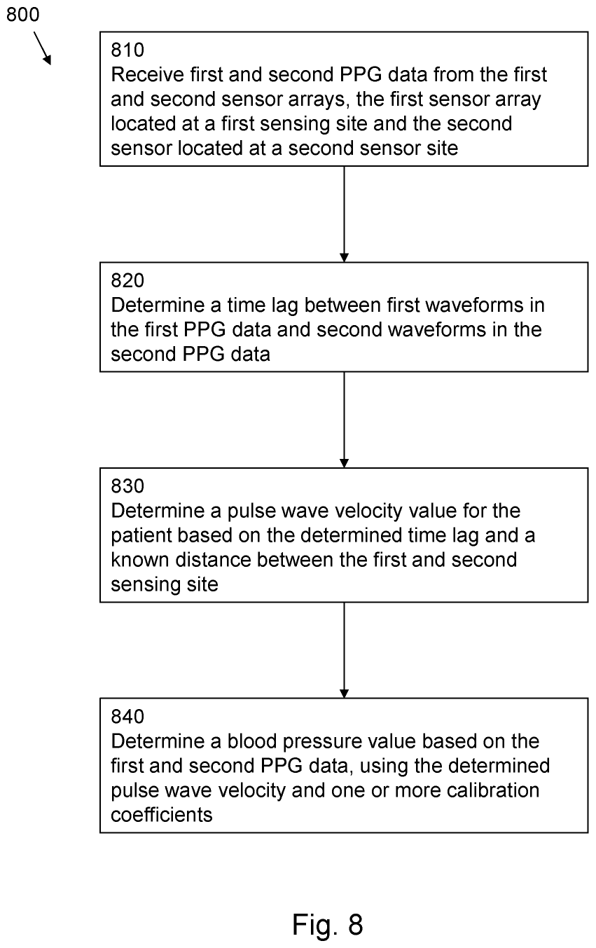

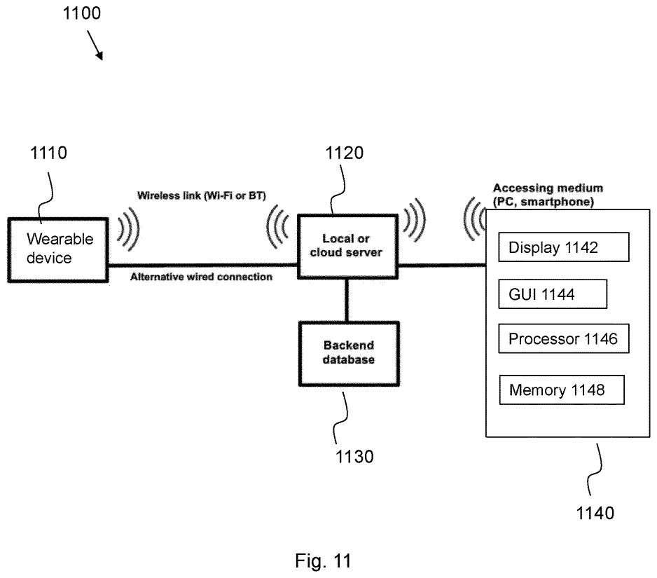

FIG. 8 depicts a method 800 according to the present disclosure, which may be performed by a processor positioned, for example, in the acquisition unit 630 or the computer 640 of system 600. With reference to FIG. 11, the method 800 may be performed by one or more processors positioned anywhere in system 1110.

At block 810, first and second PPG data is received. The first sensor data is received from the first sensor array located at a first sensor site. For example, this sensor array may be attached to a patient's wrist via a wrist strap. The second sensor data is received from the second sensor array located at a second sensor site. For example, the second sensor array may be attached to a patient's finger via a ring or other finger attachment means. While the examples of wrist and finger are used often herein, these are not the only examples and the these sensor sites are not essential. The sensor sites are positioned so that a time lag between a pulse measured at the first sensing site and the same pulse measured at the second site may be measured by the first and second sensor array. The PPG data may be of the form, or similar in form, to that depicted in FIG. 1. The PPG data may take the form of waveform data, and may be represented as one or more waveforms.

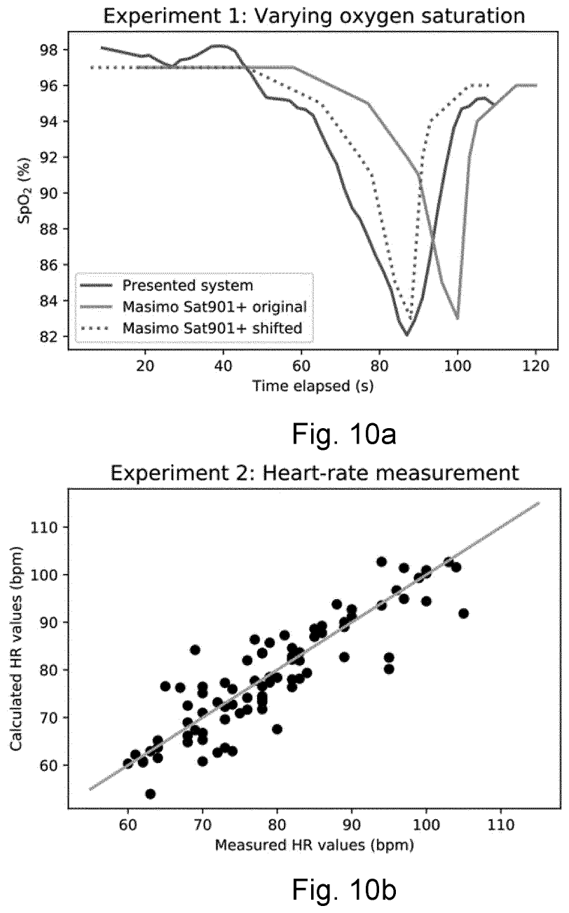

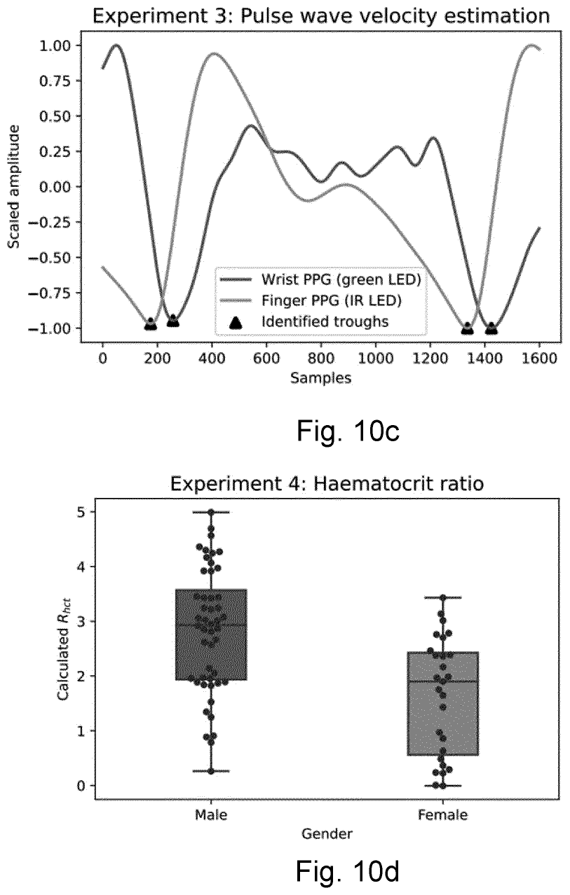

At block 820, a time lag between first waveforms in the first PPG data and second waveforms in the second PPG data is determined. The waveforms in the PPG data are representative of the patient's pulse, and the time taken for a pulse to pass from the first sensor site to the second site depends on a number of factors, including the distance the pulse must travel between the two sensor sites and the pulse wave velocity of the pulse. By measuring the time lag, and using a known, e.g. a measured or calibrated, distance between sensor sites, the pulse wave velocity may be inferred or otherwise determined. An example method for determining the time lag is described below with respect to FIG. 10c.

At block 830, a pulse wave velocity, which may be referred to as a pulse wave velocity value, for the patient is determined based on the determined time lag and a known distance between the first and second sensing site. An example method may make use of the formula speed=distance/time, for example.

At block 840, a blood pressure value is determined. The blood pressure value is determined based on the first and second PPG data, and in particular using the determined pulse wave velocity in addition to one or more calibration. A suitable formula for determining the blood pressure value is given above in the section titled “Estimation of biological parameters from PPG”.

The method may further comprise determining, based on the first and second photoplethysmography data, a haematocrit value for the patient (not shown in FIG. 8).

The method may further comprise monitoring the determined blood pressure value. For example, method 800 may be performed regularly. The result is a time series of blood pressure values associated with the patient. This monitoring may not only be limited to the patient's blood pressure, but also to one or more health parameters comprising blood pressure, haematocrit value, heart rate, and oxygen saturation in the blood.

Values for blood pressure and haematocrit are not subject to much variance in normal conditions. These can be measured at the time of enrolment (e.g. at a time when a system or device according to the present disclosure is provided to the patient) and provided they are not elevated to signify severe sickness they are used as a baseline. A simple thresholding using absolute changes may then be used to track patient progression. Each parameter may be monitored separately according to its own threshold value.

According, it will be appreciated that the method may comprise comparing the one or more health parameters to a baseline value, where the baseline value is associated with either a particular illness, pathogen and/or ailment, or associated with the patient's particular baseline value. If the one or more health parameters differs from the relevant baseline by more than a threshold value, it is determined that the change is clinically significant. Accordingly, an alert may be triggered, either to the user via their phone (e.g. via component 1140 in FIG. 11) or to a healthcare professional.

According to another implementation, data from a plurality of patients may be collected, for example, from a plurality of wearable devices each comprising a first and a second sensor array, where these devices are distributed amongst multiple patients. In this implementation, trends of patient data can be collected and acted upon. For example, the behaviour of the first and second PPG data and/or one or more of the various healthcare parameters may be examined before patients experience a clinically significant healthcare event. This data can be sued to train a machine learning model to take make more complex decisions than just triggering alerts, for example, following a thresholding process.

Generally speaking, the diagnosis of dengue shock and recurrent is made clinically by combining different aspects including abnormal vital signs, increased hct. As a rule of thumb, systolic blood pressure below 90 mmHg and rapid increase in haematocrit are indicative of patient entering shock. Anything in between can be used to track trends and issue alerts.

### Other Sensors

The present system may include additional sensors.
1. Temperature sensor: One of the early warning signs of dengue shock is cold peripheries (fingers, hands). The wearable system disclosed herein may therefore comprise a sensor configured to determine a temperature change, in particular a relative temperature change, in addition to the PPG data.

In an example use case in which a patient may have dengue, then the PPG data can be used to monitor appropriate biomarkers and identify when the patient's symptoms are worsening. Incorporation of a temperature sensor in addition to this data allows a determination of whether the patient's temperature at, for example, on or more of their extremities is decreasing. This allows a more accurate prediction of whether the patient's symptoms are worsening. In such cases, an alarm or trigger can be raised.

Accordingly, the presently disclosed system may comprise a temperature sensor configured to monitor a temperature of the patient. The processor may be further configured to determine if the patient's temperature changes from a baseline temperature for the patient by a threshold amount. If the temperature changes by the baseline amount, and/or if the PPG-derived biomarkers change by their respective amounts, an alarm may be raised in software and/or with the patient's healthcare provider.
2. Accelerometer sensor: PPG can be noisy when high movement is present. To address this problem, the wearable system may additionally comprise an accelerometer.

The PPG acquisition system can be turned “off” when too much movement is detected, e.g. when the accelerometer detects movement above a certain threshold. The system can be calibrated to determine how much patient movement is ‘too much’, i.e. the accelerometer signals above which the PPG data becomes unacceptably noisy. By not acquiring PPG data during these periods of high activity, no useful information is lost (the noisy data gets discarded anyway) but battery life can be preserved for longer which is very important for a wearable device.

Accordingly, the presently disclosed system may comprise an accelerometer configured to monitor a motion of the patient (for example accelerometer signals which are indicative of the degree of swing of their arm). The processor may be further configured to determine if the patient's motion changes from a baseline motion for the patient by a threshold amount. If the motion increases by the baseline amount, then PPG signals/data are no longer acquired in order to preserve battery life. Signal collection may be resumed, for example, when the processor determines that the accelerometer signals have returned back under the threshold value.

### Use Case: Dengue

The present system is particularly well-suited for monitoring of the main biomarkers affected by dengue. In the unlikely case that the dengue progresses into a more severe state, the patient become at risk of dengue shock that manifests itself as plasma leakage. Plasma leakage for these patients has a direct effect on increasing haematocrit and decreasing blood pressure in a short period of time. As these modalities are constant for healthy subjects, the present system and methods present a unique opportunity to track deviations from a baseline for at risk patients as opposed to providing absolute values of vital signs. This way the requirements for accuracy of vital sign estimation can be relaxed, and a focus on tracking changes throughout the lifecycle of infection can be focused on.

In a system and method specifically configured for monitoring of biological parameters affected by Dengue, the system may be configured to determine if a patient's haematocrit has increased by a first threshold amount (whether an absolute or percentage threshold) from their haematocrit baseline. The system may be further, or alternatively, configured to determine if a patient's blood pressure has increased by a second threshold amount (whether an absolute or percentage threshold) from their blood pressure baseline. If one or both thresholds are reached, the system may send an alarm, notification, or otherwise alert the patient or healthcare authorities. This prompt alert using real-time measurements enables the patient to seek medical attention in a prompt and efficient manner.

In addition, as discussed above, the patient's temperature may also be monitored with respect to a baseline temperature for the patient. It maybe determined whether or not the patient's temperature decreases below a threshold value with respect to that baseline. If one or all thresholds are reached (haematocrit, blood pressure, temperature), the system may send an alarm, notification, or otherwise alert the patient or healthcare authorities. This prompt alert using real-time measurements enables the patient to seek medical attention in a prompt and efficient manner.

### Use Case and Experimental Data

To give an example implementation of the presently disclosed system and methodologies, a particular and specific experiment will be described. The skilled person will understand that the present system and methodologies have far wider areas of applicability and many more use cases are possible. To verify the usability and accuracy of the proposed platform, a healthy volunteer study was conducted at Imperial College London. The study in total involved 10 participants (4 female, 6 male) aged 23-32. Data were acquired from both sensor probes on one hand of each subject's right hand. A laptop was used to visualise the data in real time and save the data locally. Measurements were obtained from each subject's other hand using a clinical-grade pulse oximeter (in this case, a Masimo Sat901+). A highest supported sampling frequency of 1 kHz was chosen. When sampling at this frequency, the resultant file size for all 8 channels together is approximately 12 MB per 1 minute of recording. While significant, the modern SSDs are fast and large enough to support multi-hour continuous recordings.

### A. Example Waveforms

As the two sensor probes operate independently, data timestamps for synchronisation are processed before signal processing starts. In most cases, the AFE4900EVM boards internal oscillators are not matched causing difference in sampling rates of up to 12 Hz between the two boards when acquiring data at 1 kHz. To combat this, the true sampling rate is extracted from saved timestamps during post-processing and waveforms from one board are resampled to match frequency with the other. Using their respective starting timestamps the two boards are then synchronized together to a common starting point for all acquisition channels. The system was validated by a series of experiments, recording both sensor probes across all available wavelengths. Throughout the experimental section, the raw signal is analysed using a unified signal processing pipeline.

With reference to the signal processing pipeline depicted in FIG. 7, the pipeline used in this experiment had 5 stages:
1) Resampling stage: Data from wrist probe are resampled to match frequency of finger probe and synchronized into a single large matrix.
2) Low-pass stage: Data is filtered with a Butterworth filter with cutoff frequency of 20 Hz. At this point an alternative snapshot of the dataset is saved, to be used for DC part of the AC/DC ratio calculation in pulse oximetry.
3) High-pass stage: Employing a high-pass Butterworth filter with 0.5 Hz cutoff to removes DC offset from the signal. Snapshot of the dataset is saved again, to be used for the AC part of the AC/DC ratio calculation in pulse oximetry.
4) Scaling stage: The signal is scaled between 1 and 1, pronouncing features and allowing consistent peak detection for heart-rate and PWV analysis.
5) (Optional) Smoothing stage: Employing s Savinsky-Golay filter, this stage allows reconstruction of low amplitude PPG suffering from non-periodic noise.

The final pipeline output scaled and normalized the waveform to allow precise feature extraction and accurate peak detection. For SpO2 or other ration based calculations, the intermediate results of the pipeline are used instead.

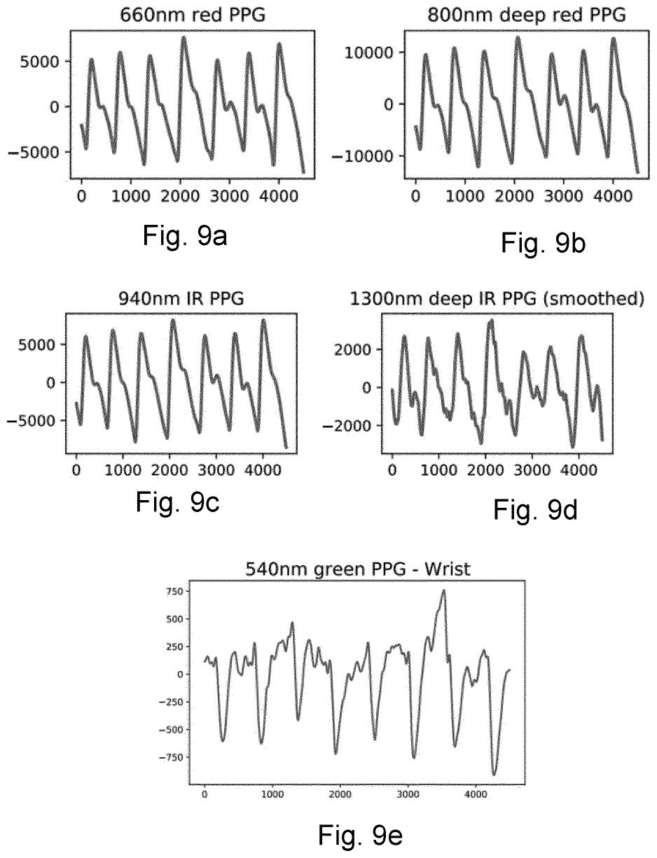

FIGS. 9a-d depict example filtered waveforms collected during this experiment, including example filtered waveforms acquired from the finger sensor probe at all 4 wavelengths. For the deep IR wavelength at 1300 nm, water starts to dominate the absorption spectrum, significantly attenuating the transmitted signal and introducing noise. After smoothing the waveform using the Savitzky-Golay filter, the waveform becomes recognisable and both the AC trends are sufficient for extraction of high-level parameters.

FIG. 9e depicts an example waveform acquired from the wrist sensor probe. The green wavelength has a larger amplitude variation thanks to the lower light penetration depth which results in shorter light path throughout tissue and smaller attenuation of the transmitted signal.
1) Finger recordings: The finger sensor probe provided clear PPG at 660 nm, 800 nm and 940 nm after passing through the signal processing pipeline. At 1300 nm, the water becomes the dominant absorbing medium and due to large amounts of water surrounding the arteries in human body, the transmitted light signal is heavily attenuated. The resultant waveform may be reconstructed using a Savitzky-Golay smoothing filter as shown in FIGS. 9a-d. FIGS. 9a-d illustrate the difference in waveform between the four fingertip wavelengths.

After reconstruction, the deep IR waveform provides recognisable peaks at the dominant frequency and can be used for ratio-based calculations and peak detection. For algorithms where more precise features like dicrotic notch or area under the curve are required, the clean PPG from other wavelength channels are used.
2) Wrist recordings: The addition of the second sensing location allows the system to extract the time difference between finger and wrist waveform, provided both contain discernible peaks.

Due to the increase in noise corruption caused be the reflectance method of waveform acquisition, there is a clear difference in waveform shape between finger and wrist. The green (525 nm) channel can be used, as its lower tissue penetration depth makes it less susceptible to noise and in turn more robust for peak detection. The observed waveform in FIG. 8e shows clearly defined peaks corresponding to heart-rate allowing the PWV estimation.

### B. SpO2 Calibration Experiment

The commonly used empirically derived equation relating the red and infrared wavelength ratio and SpO2 is defined as:

SpO₂=110−25*R (4)

where R has been previously defined in equation 2. A trained experiment subject can momentarily decrease his or her oxygen saturation in their blood by varying their breathing rate, allowing a comparison of the accuracy and sensitivity of the present system with a medical grade pulse oximeter. Throughout the experiment, continuous values from both sensors were recorded and the resultant SpO2 values are plotted in FIG. 10a. FIG. 10a shows varying oxygen saturation, comparing the presently disclosed system with the clinical grade clinical-grade pulse oximeter results (both shifted and not shifted).

It is important to note that the clinical-grade pulse oximeter results only show rounded SpO2 values, and as observed during the experiment it has a delayed response. The subject was already breathing normally by the time the oxygen saturation started dropping. The observed time delay was around 10 s and it corresponds to the shift shown in FIG. 10a. The delay is attributed to the clinical-grade pulse oximeter device software.

In terms of absolute values, once shifted in time, the mean error in SpO2 value is 2.51% with a standard deviation of 2.11. The guidelines for performance of medical pulse oximeters by the U.S. Food and Drug Administration (FDA) requires the measured value to be within 4% of the true value obtained invasively. These results show that the measured ratio between PPG waveforms follows the changes in oxygen saturation in blood and therefore the system is capable of pulse oximetry.

### C. SPO2 And Heart-Rate Measurement

The remaining experiments use a dataset that has been collected as a part of the clinical study on healthy volunteers at Imperial College London. Each session was 10 minutes long and recorded with participants sitting down and at rest. For PWV calculation, the distance between the two probes was measured for each participant at the start of the session. Every minute throughout the recording, a spot measurement of heartrate and oxygen saturation was taken from the pulse oximeter. To calculate the corresponding HR and SpO2 values from acquired raw waveforms, a 5 second segment was isolated at the start of each minute for every subject, leading to 10 datapoints per subject. In some cases, the waveform segment around the 1 minute mark was suffering from motion artefact noise and therefore was omitted.

Overall, 84 points were analyzed and their comparison to the clinical-grade pulse oximeter values is illustrated in FIG. 10b. The achieved mean error was 4.08 bpm with a standard deviation of 3.72. It is important to note that the Experiment 1 (SpO2 calibration experiment) demonstrated a time lag between values from the clinical-grade pulse oximeter device and real-time continuous values from the presented system. Combined with the variance in subjects heart-rate of up to 20 bpm over the course of a single 10 minute recording, the measured error is within the expectation. The data show that the present system is capable of continuous hear-rate monitoring across the healthy subject cohort.

Obtained SpO2 measurement results are illustrated in the table I. For one subject in particular, the empirical equation (4) does not provide adequate results with close to 5% mean error. However, the low standard deviation indicates that error was mostly caused by a constant offset. The remaining 9 subjects achieved the mean error of 1.54% which resides within the FDA allowed limits.

While these results indicate that the empirical equation (4) requires refinement prior to its application across the general population, the experiment confirms that the platform produces valid ratio R for the two pulse oximetry wavelengths across the experimental cohort.

TABLE 1
Table of SpO2 measurement results in healthy volunteers.

| | Across | Excluding | 10th subject |
|---|---|---|---|
| | all | 10th subject | only |
| | (%) | (%) | (%) |
| Mean error | 1.85 | 1.54 | 4.86 |
| Standard deviation | 1.41 | 1.04 | 0.82 |

### D. Analysis of Pulse Wave Velocity (PWV) as a Precursor for Blood Pressure Estimation

The FIG. 10c illustrates an approach for extracting the time lag between the waveforms from the first and second sensor array, e.g. from the wrist and finger waveforms. The troughs of both waveforms are identified using a predictive peak detector and the difference between them is computed across the whole recording. A single recording is split into 10 1-minute segments. For a segment to be deemed applicable for PWV extraction, over 10% of the peaks in the same region within both synchronised signals need to be of sufficient quality. Due to the nature of varying shape and noise levels within PPG waveform, and a lack of general DSP tools that can be applied in every situation, the final quality checks were done manually. Sufficient quality for fingertip signal was defined as PPG shape with no deformities including dicrotic notch as illustrated in FIG. 1.

For the wrist signal, the quality was deemed sufficient in the case where each period was clearly defined by two troughs as shown in FIG. 9e. The participant was only scored if at least 50% of segments evenly split across the 10 minute recording were applicable for PWV extraction. It is possible to define the relationship between PWV in m/s and trough position difference (5) as:

$$PWV = \frac{a}{δ + x} \times 1 \; 000 \qquad ( 5 )$$

Where d is the finger-wrist distance in meters, δ is number of samples between the troughs when sampled at 1 kHz and x is constant offset introduced by hardware limitations. Assuming the PWV values within our cohort correspond to known reference values (see e.g. A. Diaz, C. Galli, M. Tringler, A. Ramirez, and E. I. Cabrera Fischer, “Reference Values of Pulse Wave Velocity in Healthy People from an Urban and Rural Argentinean Population,” International Journal of Hypertension, vol. 2014, 2014) the constant offset x for each participant can be calculated. The normalized value PWVn can then be obtained by subtracting the offset x from mean δ for each participant and recalculating PWV. The table II summarizes obtained results. Table II shows a summary of the PWV experiment where PWVn is the normalized PWV value after removing offset x, σPWVn is a standard deviation of PWVn, d is finger-wrist distance and x is the random offset introduced by hardware when starting the recording.

TABLE II
Summary of PWV experiment

| | Finger-wrist | PWVₙ ± σ_{PWV}_{ₙ} | x offset | No. of beats |
|---|---|---|---|---|
| ID | distance (cm) | (m/s) | (samples) | analyzed |
| 1 | 17.1 | 7.31 ± 3.60 | 87.90 | 53 |
| 2 | 15.9 | 5.45 ± 0.81 | 8.17 | 131 |
| 3 | 21.0 | 5.53 ± 1.32 | 121.49 | 138 |
| 4 | 20.5 | 5.52 ± 0.84 | 21.61 | 80 |
| 5 | 20.7 | 6.66 ± 2.09 | 16.65 | 76 |
| 6 | 18.5 | 6.54 ± 2.24 | 45.74 | 141 |
| 7 | 18.1 | Wrist data too noisy | N/A | N/A |
| 8 | 16.9 | Wrist data too noisy | N/A | N/A |
| 9 | 20.9 | 5.44 ± 1.19 | 169.17 | 180 |
| 10 | 20.0 | Wrist data too noisy | N/A | N/A |

While steps were taken to reduce synchronization error, the black box implementation of the TI development board and parallel serial port communication introduces a random delay x between the two boards every time a new recording is started. It is important to note that this time delay is constant throughout each recording and therefore can be eliminated with initial calibration of the system.

Looking more closely at the results in table II, a significant difference can be noted in the calculated value between the first participant and the rest of the cohort. With only 53 individual beats analyzed, this participant was on the edge of acceptability for PWV extraction. Coupled with issues of peak definition even for peaks marked as “good” that introduces variations in the final peak position, we have excluded this participant from the overall summary. The overall weighted average PWV after normalization was 5.80±1.58 m/s matching with the values found for this age group in the literature and showcasing the capability of the system to measure the baseline time difference between the two waveforms.

### E. Haematocrit Ratio

FIG. 10d shows a plot of the Haematocrit ratio (Rhct) to illustrate a difference between values obtained from male and female participants.

The Hct ratio results are raw, without any significant post-processing involved. By calculating the ratio between 800 nm and 1300 nm wavelength as defined by equation 2 we can calculate an average Rhct for each 1 minute segment within the 10 minute recording. Repeating the approach for every participant, removing clear outliers due to noisy data and separating by gender, the results of FIG. 10d may be derived. Based on the available literature, the reference values for haematocrit are 40-54% for men and 36-48%. It is notable that the obtained Rhct values for female participants do overall differ from male values. The median value of Rhct was 2.93 and 1.90 for male and female respectively. The male median value lies outside of the female upper quartile boundary by 0.5 which points to a likely difference between the two distributions.

### Advantages

A completely modular PPG acquisition platform is described herein, allowing continuous, synchronized acquisition from a plurality of different locations with multiple (e.g. 4) LED wavelengths each. In addition, the application describes a pair of probes for two different sensor sites, in particular wrist and finger sensing, which is particularly suitable for monitoring patients suffering from dengue. The wavelength in each probe has been selected such that modalities that correlate with severe dengue can be measured. In a series of experiments, we have shown the capability of the system to measure these modalities on a limited number of subjects in lab environment.

Results obtained using the system prove the platform is capable of heart-rate monitoring, oxygen saturation monitoring, and is able to calculate a time delay between a finger and wrist waveform as well as adequate deep infra-red PPG waveform at 1300 nm. When compared with the clinical-grade pulse oximeter medical device, a mean error of 4.08±3.72 bpm for heart-rate and 1.54±1.04 for SpO₂ have been achieved. Taking into consideration the synchronisation offset between the clinical-grade pulse oximeter device and our real-time data, these results showcase that the presented system sensing capabilities are on par with those of a clinical-grade medical device. Furthermore, the PWV experiment showed that the system is capable of recording the time offset between two synchronized probes despite the hardware imperfections introducing an additional delay. The calculated R_{hct} that serves as a surrogate for haematocrit estimation has shown significant difference between male and female values as supported by relevant literature on hct values in healthy population.

The calculation of a haematocrit value in the manner described and using the present system is a significant improvement on the prior methods and systems, which typically make use of a blood sample, a centrifuge, and requires significant wait times between sample collection and a result.

The system may be a fully wearable platform. Low-cost and off-the-shelf availability of the components enables device mass-production and deployment for largescale patient management in the affected regions. With the recent shifts in medicine to remote patient monitoring and individually-tailored treatment, the present platform is able provide richer data than conventional PPG systems to help identify specific adverse effects during infectious disease outbreaks.

### Machine Learning Algorithm

It will be understood that the above description of specific embodiments is by way of example only and is not intended to limit the scope of the present disclosure. Many modifications of the described embodiments are envisaged and intended to be within the scope of the present disclosure. For example, while the above disclosure describes the determination of biological parameters using algorithms, the present inventors have realised that it is also possible to use machine learning techniques.

### Input Dataset

In an example machine learning approach for hematocrit classification, a training data set is first obtained. For example, hematocrit estimation is done solely from the fingertip probe described herein, using all 4 LED channels to estimate a hematocrit value of the blood. The input is four independent PPG waveforms with various wavelengths (660 nm, 800 nm, 940 nm, 1300 nm) sampled at identical frequency of 1000 Hz and down sampled to 100 Hz for analysis as there are no high frequency features required for this estimation.

The four channels of raw photodiode output are processed through a digital signal processing pipeline, before being transformed into an input for a suitable machine learning model.

### Pre-Processing Pipeline

The down sampled waveforms may be passed through a Chebyshev type II low pass filter with cutoff frequency at 20 Hz followed by high pass filter with cutoff at 0.5 Hz. This is a standard filtering approach for PPG signals as outlined in literature (e.g. Liang, Y., Elgendi, M., Chen, Z. et al. An optimal filter for short photoplethysmogram signals. Sci Data 5, 180076 (2018). https://doi.org/10.1038/sdata.2018.76)

The waveforms then pass-through a quality check using a mixture of SQl evaluation and heartpy Python library analysis. Once deemed good quality for further analysis, each channel is segmented into one minute or longer segments that are transformed into inputs for the machine learning model.

### Feature Extraction

Well established time series data analysis techniques may be used, for example using STFT (short-time fourier transforms) and/or convolutional neural networks. Segments are transformed into STFT spectrogram by applying consecutive Fourier transforms to create a 2D image. For each segment, four images are created, one for each LED channel. These are then stacked together and treated as a single sample.

Labelling of samples is done using clinical information acquired using known techniques. For example, each stacked image consisting of all 4 LED channels is assigned a hematocrit value as per invasively obtained values in the clinical file.

### Model Architecture

A CNN model consisting of a given amount of convolution and pooling layers is trained and evaluated for classification. A suitable train-test split in this case is a standard 80-20. The classes in this case are ranges of hematocrit values (eg. Class 1: hct 30-35%, Class 2: hct 35%-40%, Class 3: hct 40%-45%, etc.). The optimal ranges and number of classes with the best performance may be determined for different use cases.

At the end of training and cross-validation, the model can be used for real-time classification of recording (once the recording has been long enough so that the first segment becomes available). Provided the data is of sufficient quality, the output of the model determines which class does the analyzed segment most likely belongs to.

Accordingly, it will be appreciated that the haematocrit value and other biological parameters can be determined from data collected using the presently disclosed system using machine learning algorithms and techniques.

The methods described herein, based on machine learning principles or otherwise, may be performed on a chip or any suitable processor within the housing of a fully-contained wearable system. A suitable processor may take the form of an application-specific integrated circuit (ASIC) or other integrated circuit (IC) chip. The first and second sensor array may be communicatively coupled to the IC chip to enable processing of the signal on a wearable device, without the need for communication with an external data acquisition unit or computer.

A wearable system comprising both sensor arrays and a suitable processor, comprising an on-chip classifier using the machine learning approach described above, will prevent the transfer of large amounts of data over a wired or wireless connection, and will enable the transfer of a determination or classification hen action needs to be taken, e.g. if a particular biological parameter being monitored deviates from a patient's baseline by a particular threshold amount.

### FIG. 11

FIG. 11 depicts a system 1100 according to the present disclosure. System 1100 is a photoplethysmography (PPG) system for health monitoring. The system 1100 comprises a wearable device 1110, a local or cloud server 1120, a backend database 1130, and an accessing medium 1140.

The wearable device 1110 comprises a first and a second sensor array (not shown) in the manner described elsewhere herein; and in particular comprises (not shown) a first sensor array configured to collect first PPG data and configured for attachment to a patient at a first sensing site, and a second senor array configured to collect second PPG data and configured for attachment to the patient at a second sensing site.

The system also comprises one or more processors. The one or more sensors are coupled to each of the first and second sensor arrays. This coupling may be via a wired connection, or a wireless connection. “Coupling” in this sense should therefore be taken to mean a communicative coupling. The one or more processors are configured to perform the methods of the present disclosure, and in particular are configured determine, based on the first and second PPG data and a known distance between the first and second sensing site, a blood pressure value for the patient.

The wearable device 1110, which comprises multiple components and therefore may also be referred to as a wearable system, is configured to be worn on the user's arm or wrist. The system has a built-in battery (not shown) to allow wireless operation. The system 1110 further includes: sensing probes from two locations, an analogue front-end chip guiding PPG acquisition, microcontroller (processor) to aggregate and process acquired data and to facilitate wireless or wired communication with the server.

The server 1120 is a local (physically close, on-premises) or cloud (online) computing system that can be accessed via wireless (wi-fi or Bluetooth) or wired connection. In the case of a cloud server, the wired connection is a proxy device that will in turn stream the data online. The server implements a backend database service via communication with backend database 1130, that aggregates data from all the wearable devices 1110 in operation. Only a single wearable device 1110 is shown in FIG. 11, however it should be appreciated that, according to some implementations, a plurality of wearable devices 1110 are in communication with server 1120. Further to this, the server 1120 allows more computationally intensive algorithms to run and therefore machine learning algorithms on the data are likely to be run server-side rather than using the limited processing power on the wearable device 1110.

In implementations involving comparing biological parameters to one or more baselines, this monitoring process may be performed on the server to make use of a greater degree of computing power available compared with the smaller on chip processor.

The system 1100 further comprises a data accessing device 1140 such as a PC, tablet or smartphone. The data accessing device 1140 can be any device supporting web applications. In the case where the server 1120 is a PC running on-premises, the data-accessing application can also run on the server 1120 itself. A suitable web application is a web-based front end able to fetch data from the server-side database 1130 and show an overview for each patient. Users, for example healthcare professionals, can further use the app to browse through the patients, export data reports and receive alerts on problematic patients.

Accordingly, with reference to FIG. 8, the method 800 may be performed by one or more processors positioned anywhere in system 1110.

In a first example, the server 1120 may receive PPG data from the wearable device at step 810. The server 1120 may then determine, based on the first and second PPG data and a known distance between the first and second sensing site, a blood pressure value for the patient; for example, server 1120 may perform steps 820, 830 and 840. The server 1120 may additionally calculate other biological parameters using the PPG data according to methods disclosed herein or else that will be known the skilled person. The server 1120 may then save these parameters on the back-end database 1130, and provide access to this information to a suitably authorised user via a web application accessed via a user's phone 1140.

In a second example, a microprocessor of the wearable device 1110 receives the PPG data (step 810) and additionally determines a time lag between the waveforms embodied within first PPG data and waveforms embodied within second PPG data (step 820). The wearable device then transmits this information, e.g. the PPG data and determined time lag, to the server 1120 to allow the server 1120 to perform steps 830 and 840.

Finally, in a third example, a microprocessor positioned within wearable device 1110 may perform method 800 in its entirety, and communicate either all the information (PPG data, time lag, pulse wave velocity and blood pressure value) to the server 1120 for storage in the backend database 130, or may simply transmit the determined blood pressure value to the server 1120 so as to reduce the amount of information to be transmitted.

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein. This computer-readable medium may be, for example, arranged within the wearable device, the server 1120, the accessing medium 1140, or any combination of these components.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

FIGS. 3, 6 and 11 illustrates example block diagrams of implementations of a system within which a set of instructions, for causing the computing device to perform any one or more of the methodologies discussed herein, may be executed. The skilled person will appreciate that alternative implementations exist. The term “processor” shall also be taken to include any collection of processors (e.g., computers, microprocessors, mobile devices, servers etc.) that individually or jointly execute a set (or multiple sets) of instructions to perform any one or more of the methodologies discussed herein.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying”, or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A photoplethysmography (PPG) system for health monitoring, the system comprising:
a first sensor array configured to collect first PPG data and configured for attachment to a patient at a first sensing site;
a second sensor array configured to collect second PPG data and configured for attachment to the patient at a second sensing site; and
one or more processors configured to receive the first PPG data and the second PPG data and determine, based on the first PPG data, the second PPG data, and a known distance between the first sensing site and the second sensing site, a blood pressure value for the patient.

2. The PPG system of claim 1, wherein determining the blood pressure value for the patient is further based on a time lag between first waveforms in the first PPG data and second waveforms in the second PPG data.

3. The PPG system of claim 2, wherein determining the blood pressure value for the patient comprises:
determining the time lag;
determining a pulse wave velocity value for the patient based on the determined time lag and the known distance between the first and second sensing site; and
determining the blood pressure value using the determined pulse wave velocity value and one or more calibration coefficients.

4. The PPG system of claim 1, wherein the first sensor array is configured to collect the first PPG data in a first plurality of wavelength channels; and
the second sensor array is configured to collect the second PPG data in a second plurality of wavelength channels.

5. The PPG system of claim 4, wherein the first plurality of wavelength channels and the second plurality of wavelength channels each comprise at least three wavelength channels.

6. The PPG system of claim 4, wherein the second plurality of wavelength channels comprises at least two wavelengths which define an isosbestic point for Haemoglobin (HB) and Oxy-haemoglobin (HbO2).

7. (canceled)

8. The PPG system of claim 1, wherein the one or more processors are further configured to determine, based on the first PPG data and the second PPG data, a haematocrit value for the patient.

9. The PPG system of any claim 1, wherein the one or more processors are further configured to monitor one or more health parameters comprising blood pressure, haematocrit value, heart rate, and oxygen saturation in the blood.

10. The PPG system of claim 9, the one or more processors being further configured to compare the one or more health parameters to a baseline value for the patient and, if the one or more health parameters differs from the baseline value by more than a threshold value, trigger an alert.

11.-13. (canceled)

14. The PPG system of claim 1, wherein the system is wearable.

15. The PPG system of claim 1, wherein the one or more processors comprises a first processor and wherein the system further comprises a server;
wherein the system comprises a wearable device comprising the first sensor array, the second sensor array, and the first processor; and
wherein the first processor is configured to:
receive the first PPG data and the second PPG data; and
communicate the first PPG data and the second PPG data to the server; and
wherein the server is configured to determine the blood pressure value for the patient.

16. The PPG system claim 1, further comprising:
a temperature sensor configured to monitor a temperature of the patient; and
wherein the one or more processors are further configured to determine if the patient's temperature changes from a baseline temperature for the patient by a threshold amount.

17. The PPG system of claim 1, further comprising:
an accelerometer configured to monitor a motion of the patient; and
wherein the one or more processors are further configured to determine if the patient's motion changes from a baseline motion for the patient by a threshold amount.

18. A computer-implemented method for use with a photoplethysmography (PPG) system, the method comprising:
receiving first PPG data obtained from a first sensor array attached to a first sensing site of a patient;
receiving second PPG data obtained from a second sensor array attached to a second sensing site of the patient; and
determining, based on the first PPG data, the second PPG data, and a known distance between the first and second sensing site, a blood pressure value for the patient,
wherein the PPG system comprises:
the first sensor array, configured to collect the first PPG data and configured for attachment to the patient at the first sensing site;
the second sensor array, configured to collect the second PPG data and configured for attachment to the patient at the second sensing site; and
one or more processors configured to receive the first PPG data and the second PPG data and determine, based on the first PPG data, the second PPG data, and the known distance between the first sensing site and the second sensing site, the blood pressure value for the patient.

19. The method of claim 18, wherein determining the blood pressure value for the patient is further based on a time lag between first waveforms in the first PPG data and second waveforms in the second PPG data.

20. The method of claim 19, wherein determining the blood pressure value for the patient comprises:
determining the time lag;
determining a pulse wave velocity value for the patient based on the determined time lag and the known distance between the first sensing site and the second sensing site; and
determining the blood pressure value using the determined pulse wave velocity value and one or more calibration coefficients.

21. The method of claim 18, further comprising determining, based on the first PPG data and the second PPG data, a haematocrit value for the patient.

22. The method of claim 18, further comprising monitoring one or more health parameters comprising blood pressure, haematocrit value, heart rate, and oxygen saturation in the blood.

23. The method of claim 22, further comprising comparing the one or more health parameters to a baseline value for the patient and, if the one or more health parameters differs from the baseline by more than a threshold value, triggering an alert.

24. A computer readable medium comprising instructions which, when implemented by a processor, cause the processor to:
receive first PPG data obtained from a first sensor array attached to a first sensing site of a patient;
receive second PPG data obtained from a second sensor array attached to a second sensing site of the patient; and
determine, based on the first PPG data, the second PPG data, and a known distance between the first and second sensing site, a blood pressure value for the patient.
